(19) United States
(12) Patent Application Publication
(10) Pub. No.: US 2006/0155286 A1
(43) Pub. Date: Jul. 13, 2006

(54) Bone securing bolt

(76) Inventor: Chao-Jan Wang, Hsinchu City (TW)

Correspondence Address: BACON & THOMAS, PLLC, 625 SLATERS LANE, FOURTH FLOOR, ALEXANDRIA, VA, 22314, US

(21) Appl. No.: 11/032,003

(22) Filed: Jan. 11, 2005

(51) Int. Cl. A61B 17/58

(52) U.S. Cl. 606/73

(57) ABSTRACT

A bone securing bolt includes a threaded shank, a head formed on an end of the threaded shank, a receiving space defined in the threaded shank, multiple through holes defined in a side face of the threaded shank to communicate with the receiving space, and multiple blind holes defined in the side face of the threaded shank.

### BACKGROUND OF THE INVENTION

1. Field of the Invention

The present invention relates to a bolt, and more particularly to a bolt for securing a bone.

2. Description of Related Art

When a patient has a bone fracture, doctors will use a bone securing bolt to perform an appropriate operation. That is, the bone securing bolt will be applied to connect two pieces of the bone to allow the broken bone pieces to connect in growth. The conventional bone securing bolt (6) is shown in FIG. 6. The bone securing bolt (6) has a threaded shank (61) and a head (62) integrally formed at one end of the threaded shank (61). Furthermore, a securing plate (7) is applied to have multiple through holes (71) defined through the securing plate (7) to correspond to multiple securing bolts (6). When the conventional bone securing bolt (6) and the securing plate (7) are used as shown in FIGS. 7 and 8, the securing plate (7) is first placed on top of the damaged bone, and then the bone securing bolts (6) are inserted into the through holes (71) and then screw into the damaged bone to secure the damaged bone pieces. However, after the bone securing bolts (6) are inserted into the bone, during the bone healing process, the newly grown bone will gradually embrace the threaded shank (61). This will prolong the patient's suffering when the doctor is removing the bone securing bolt (6) from the healed bone. That is, the doctor will have to re-open the wound to have access to and remove the bone securing bolt (6). This invasive treatment does have the expected effect to re-connect the fractured bone, but a second injury to the patient is caused due to the re-opening process for retrieval of the bone securing bolts (6).

Furthermore, with reference to FIG. 9, another conventional bone securing device is shown and has a base (a), two extensions (b,c) extending out of the base (a) and two helical positioning rings (d,e) respectively mounted on a corresponding one of the two extensions (b,c). With such an arrangement as shown in FIG. 10, a patient with osteoporosis is not able to use this kind of design, because the helical positioning rings (d,e) will deteriorate the quality of the patient's bone.

To overcome the shortcomings, the present invention tends to provide an improved bone securing bolt to mitigate the aforementioned problems.

### SUMMARY OF THE INVENTION

The primary objective of the present invention is to provide an improved bone securing bolt to be permanently affixed into the fractured bone without removal such that a second injury to the patient is avoided.

In order to accomplish the aforementioned objective, the bone securing bolt of the present invention includes a threaded shank and a head formed on an end of the threaded shank. The bone securing bolt has a receiving space longitudinally defined in the threaded shank, multiple through holes radially defined in a side face of the threaded shank and multiple blind holes defined in the side face of the threaded shank such that medulla from the newly grown bone will flow into the receiving space, through holes and the blind holes to affix the bone securing bolt in the bone. Therefore, there is no need to remove the bone securing bolt after the fractured bone is healed.

Other objects, advantages and novel features of the invention will become more apparent from the following detailed description when taken in conjunction with the accompanying drawings.

### BRIEF DESCRIPTION OF THE DRAWINGS

FIG. 1 is a perspective view of the bone securing bolt of the present invention;

FIG. 2 is a cross sectional view of the bone securing bolt in FIG. 1;

FIG. 3 is a perspective view of the second embodiment of the bone securing bolt of the present invention;

FIG. 4 is a cross sectional view of the bone securing bolt in FIG. 3;

FIG. 5 is a cross sectional view showing another embodiment of the bone securing bolt of the present invention;

FIG. 6 is an exploded perspective view of a combination of a conventional bone securing bolt and a securing plate;

FIGS. 7 and 8 are schematic operational views showing the application of the conventional bone securing bolt and the securing plate; and

FIG. 9 is a perspective view showing a different bone securing device.

### DETAILED DESCRIPTION OF THE PREFERRED EMBODIMENT

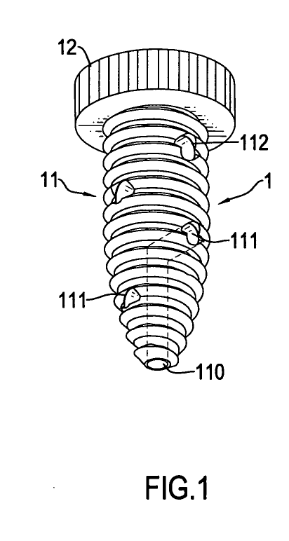

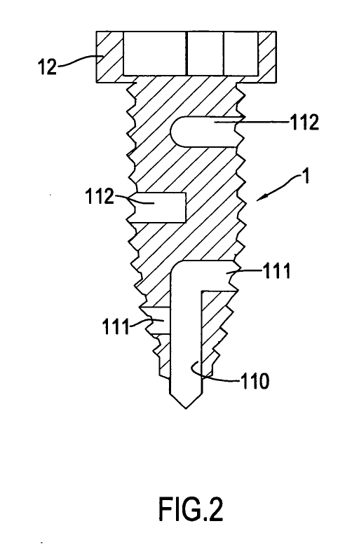

With reference to FIGS. 1 and 2, the bone securing bolt (1) in accordance with the present invention includes a threaded shank (11) and a head (12) formed on one end of the threaded bolt (11). The threaded shank (11) has a receiving space (110) longitudinally defined through a free end of the threaded shank (11), multiple through holes (111) (only one is shown in this embodiment) radially defined in a side face of the threaded shank (11) to communicate with the receiving space (110) and multiple blind holes (112) radially defined in the side face of the threaded shank (11). With such a configuration, the bone securing bolt (1) of the present invention is able to allow medulla to flow into the receiving space (110), the through holes (111) and the blind holes (112) so as to embrace the bone securing bolt (1). After the medulla from the newly grown bone has entered the bone securing bolt (1) of the present invention, any possible movement or deviation of the bone securing bolt (1) due to traction of tendons is obviated such that a second operation for retrieval of the bone securing bolt (1) is unnecessary.

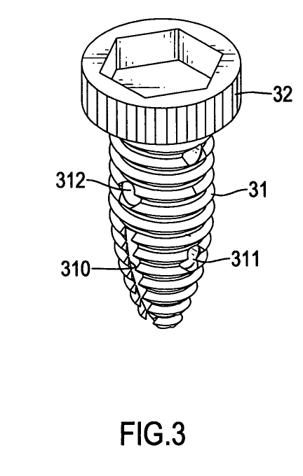

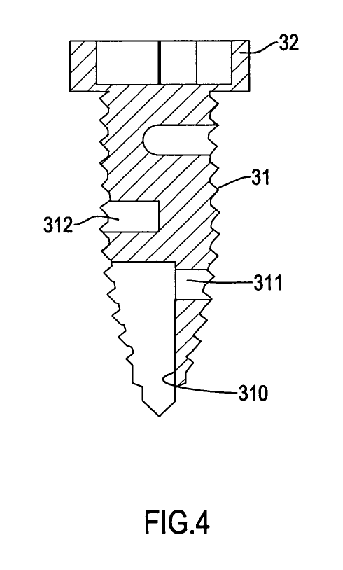

With reference to FIGS. 3 and 4, it is noted that the bone securing bolt (3) of the present invention includes a threaded shank (31) and a head (32) formed on one end of the shank (31). The threaded shank (31) has a receiving space (310) longitudinally defined through opposed side faces of the threaded shank (31), multiple through holes (311) (only one is shown in this embodiment) radially defined in a side face of the threaded shank (31) to communicate with the receiving space (310) and multiple blind holes (312) radially defined in the side face of the threaded shank (31). With such a configuration, the bone securing bolt (3) of the present invention is able to allow medulla to flow into the receiving space (310), the through holes (311) and the blind holes (312) so as to embrace the bone securing bolt (3). After the medulla from the newly grown bone has entered the bone securing bolt (3) of the present invention, any possible movement or deviation due to traction of tendons is obviated such that a second operation for retrieval of the bone securing bolt (3) is unnecessary.

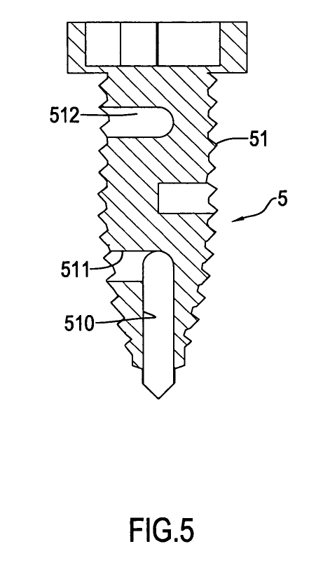

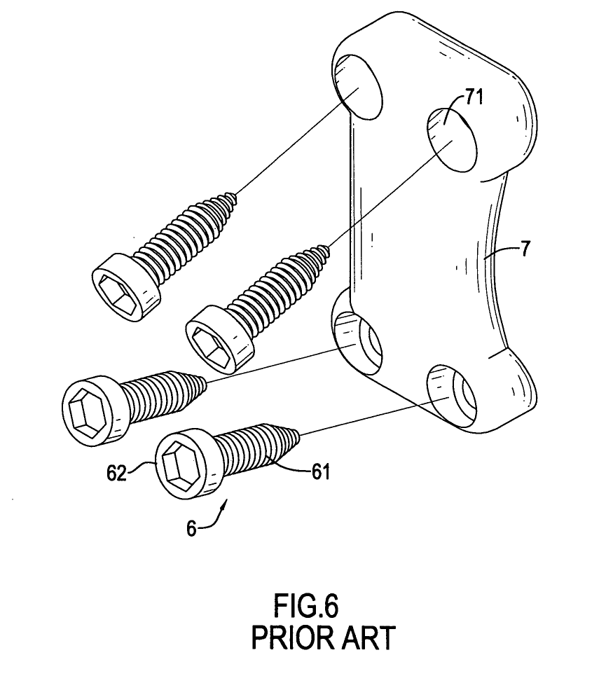

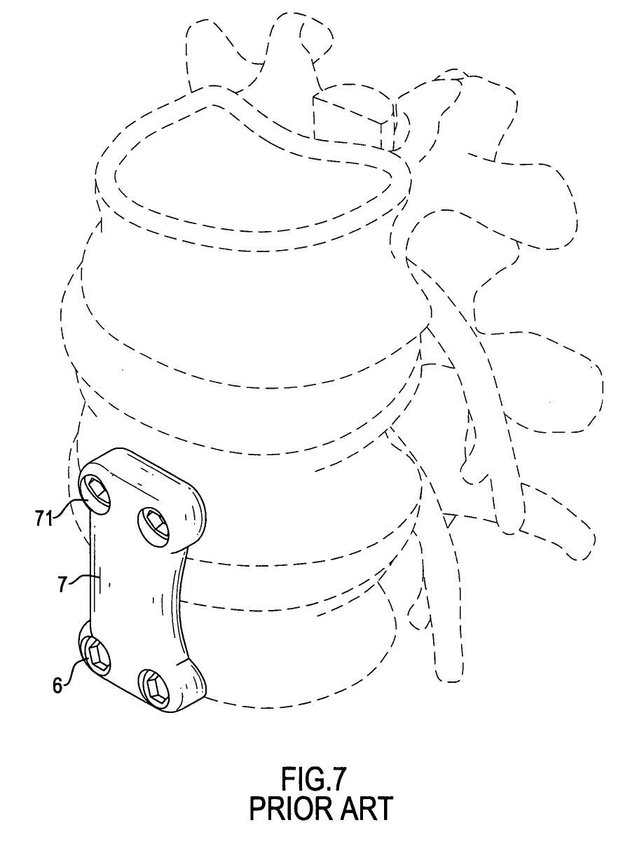

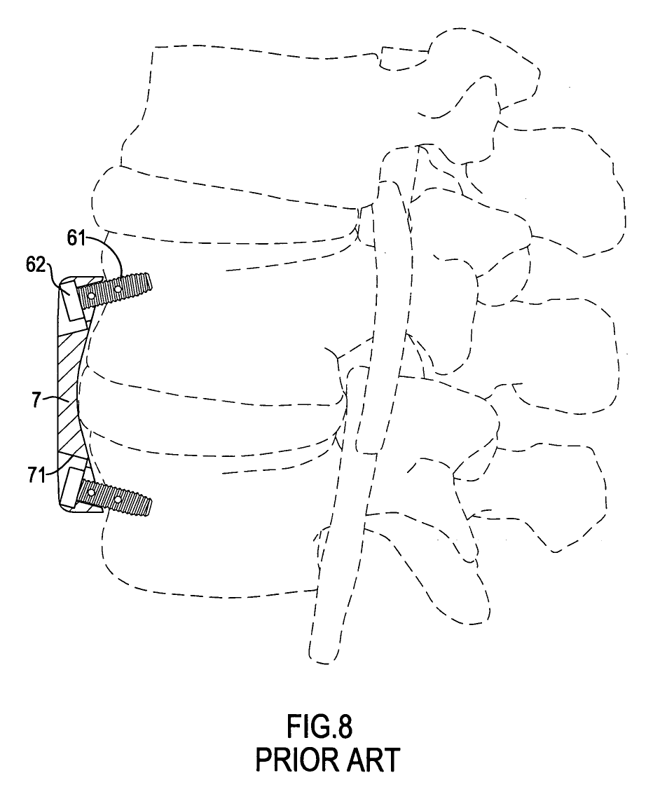

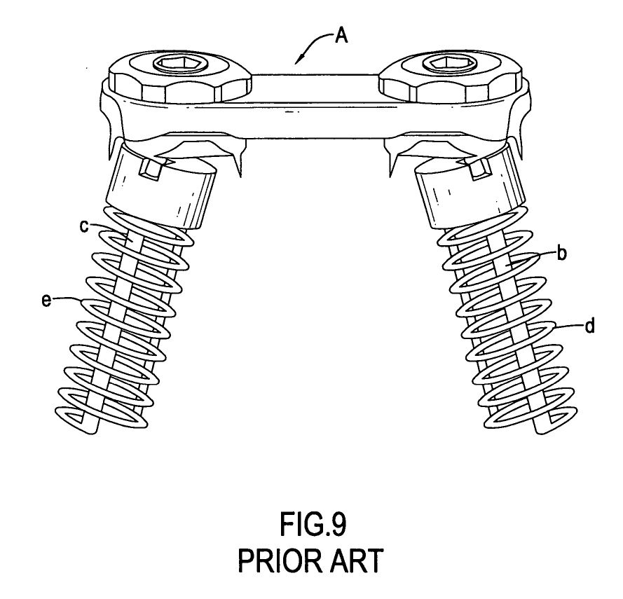

With reference to FIG. 5, it is noted that a receiving space (510) of the bone receiving bolt (5) in this embodiment does not extend through the free end of the threaded shank (51). The threaded shank (51) has multiple through holes (511) radially defined through a side face of the threaded shank (51) to communicate with the receiving space (510) and multiple blind holes (512) radially defined in the side face of the threaded shank (51).

After the bone securing bolt of the present invention is implanted into the fractured bone, there is no need for the patient to have a second invasive treatment for retrieval of the bone securing bolt because the bone securing bolt of the present invention is securely affixed onto the fractured bone due to the medulla from the newly grown bone.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A bone securing bolt comprising:
a threaded shank;
a head formed on an end of the threaded shank;
a receiving space defined in the threaded shank;
multiple through holes defined in a side face of the threaded shank to communicate with the receiving space; and
multiple blind holes defined in the side face of the threaded shank so as to allow medulla from newly grown bone to flow into the receiving space, the through holes and the blind holes.

2. The bone securing bolt as claimed in claim 1, wherein the receiving space is longitudinally defined through a free end of the threaded shank.

3. The bone securing bolt as claimed in claim 2, wherein the receiving space is longitudinally defined through opposed side faces of the threaded shank.

4. The bone securing bolt as claimed in claim 3, wherein the through holes are radially defined in the side face of the threaded shank.
